(19) United States
(12) Patent Application Publication
(10) Pub. No.: US 2016/0151440 A1
(43) Pub. Date: Jun. 2, 2016

(54) A Novel Composition of Curcumin with Enhanced Bioavailability

(71) Applicant: Aurea Biolabs Private Limited, Cochin, Kerala (IN)

(72) Inventor: Sreeraj Gopi, Cochin (IN)

(73) Assignee: Aurea Biolabs Private Limited, Cochin, Kerala (IN)

(21) Appl. No.: 14/652,492

(22) PCT Filed: Aug. 19, 2014
(86) PCT No.: PCT/IB2014/063960

§ 371 Date: Jun. 16, 2015

(30) Foreign Application Priority Data
Aug. 19, 2013 (IN) 3646/CHE/2013

(51) Int. Cl. A61K 36/9066; A61K 38/16; A61K 31/12

(52) U.S. Cl. CPC A61K 36/9066; A61K 31/12; A61K 38/168; A23L 1/221; A23V 2002/00

(57) ABSTRACT

This invention discloses a curcumin composition for increasing the bioavailability of curcumin, which consists of curcumin mixture and water extract in a ratio of 70:30. The curcumin mixture comprises curcumin dry crystals, volatile oil, fixed oil whereas water extract comprises soluble proteins, dietary fibers and carbohydrates extracted from turmeric. The composition also consists of a natural emulsifier isolated from Quillaja saponaria and lecithin. The composition increases rate of availability of curcumin as the dietary fibers holds the curcumin for long time. The soluble proteins increase the solubility of curcumin in water thus increasing absorption of curcumin in blood and the essential oil increases dispensability. The invention also discloses the method of preparation of the composition. The curcumin composition showed bioequivalence with 500 mg curcumin capsules, exhibited anti-aging, antioxidant, anti-inflammatory activities and is also effective in boosting the phagocyte mediated immunity suggesting as natural therapy for treatment of various diseases.

### TECHNICAL FIELD OF THE INVENTION

The present invention relates to a composition containing curcumin as the main ingredient in which the bioavailability of curcumin is high. More specifically, the invention relates to a novel composition containing curcumin along with soluble proteins, dietary fiber and essential oil from turmeric that increases absorption of curcumin in the blood and results in better dispersibilty upon oral consumption, which is useful for treatment of various diseases.

### BACKGROUND OF THE INVENTION

Curcumin (1, 7 bis (4-hydroxy-3-methoxy phenyl)-1, 6 heptadiene-3, 5-dione), is a principal curcuminoid of the popular Indian spice Curcuma longa commonly known as turmeric, a perennial herb of Zingiberaceae (ginger) family, which is a 3-5 ft tall bearing oblong, pointed, short-stemmed leaves and funnel-shaped yellow flowers. Turmeric is widely cultivated in India, China and other tropical countries.

Curcuminoids are natural phenols that are responsible for the yellow color of turmeric. Curcumin exists in several tautomeric forms.

Curcumin is derived from the rhizome of Curcuma longa and has been traditionally used in the treatment of skin wounds, inflammation, tumors etc. It is a powerful antioxidant with the ability to scavenge free radicals generated in the body as a result of various metabolic processes. It is also well known for its anti-inflammatory, anti-angionenic and immunomodulatory effects. Curcumin is used for treatment of various diseases like Arthritis, Gastrointestinal upset etc. Curcumin is available in the state of the art in the form of dietary supplement because of its antioxidant benefits as it provides protection against cell-damaging free radicals. However, the extent to which the human body benefits from consumption of curcumin is doubtful and limited because of poor bioavailability of curcumin.

Many formulations of curcumin have been developed and are available in the state of the art. Most of the formulations relied on the use of piperine, a component responsible for the pungency of black pepper and long pepper as an adjuvant due to its ability to inhibit the hepatic and intestinal metabolism of curcumin. However, the use of piperine to increase the absorption rate of curcumin was not successful. Preventing the metabolism alone without enhancing the solubility does not contribute much to the improved bioavailability. The use of other adjutants such as green tea or soya bean extracts was also not found to be good enough in increasing the bioavailability of curcumin.

The US patent application numbered U.S. Ser. No. 12/073,864 titled “Composition to enhance the bioavailability of curcumin” discloses a composition containing purified curcuminoid and purified essential oil for enhanced bioavailability of curcumin. The application also discloses a method to prepare a composition for enhanced bioavailability of curcumin. The composition comprises curcuminoid mixture consisting of demethoxycurcumin and bisdemethoxycurcumin and the essential oil ar-turmerone. The percentage of curcuminoid mixture used is in the range between 24% to 96% and the essential oil in the range between 4%-50%. The dosage is suitable for oral administration and is in the form of a gelatin capsule. The application also discloses the method of extraction of curcuminoid from turmeric and also a method of preparing the composition including a curcuminoid and the essential oil of turmeric. The applications also tabulate the results of bioavailability of curcumin in human volunteers. Even though the bioavailability is increased in blood, the application is silent with respect to prevention of elimination of curcumin from blood and also with respect to the stability of curcumin in blood.

The US patent application numbered U.S. Ser. No. 13/645,031 titled “Formulation of Curcumin with Enhanced Bioavailability of Curcumin and Method of Preparation and Treatment Thereof” discloses a formulation of curcuminoid with essential oil of turmeric to enhance the bioavailability of curcumin and to augment the biological activity of curcumin. The application also discloses a method of preparation of curcuminoid formulation and also oral supplementation for treatment of various diseases. The formulation of curcuminoid mixture comprises curcumin, demethoxycurcumin and bisdemethoxycurcumin and essential oil ar-turmerone. The application evidenced increased bioavailability in human volunteer. Even though the patent application confirms the enhanced bioavailability in the range between 5-16 fold, the application is silent with respect to the stability of curcumin in blood.

The PCT application numbered PCT/US2011/048090 titled “Curcumin compositions and uses thereof” describes a composition and a method of increasing the bioavailability of curcumin. The composition is suitable for modifying DNA methylation and for treatment of cancer. The composition comprises curcumin and excipient polymers like polyethoxylated castor oil, polyoxyethylene sorbitan ester and polyethylene glycol. The application described the increased bioavailability of curcumin by using gel formulation and the formulation is safe, tolerated and effective in treatment of leukemia and breast cancer. The plasma level of curcumin was high in human volunteer thus increasing the bioavailability of curcumin. However, the invention is silent with respect to the absorption of curcumin from blood and its dispersibility, which affects the bioavailability.

The major problems of poor bioavailability of curcumin are poor absorption, rapid metabolism and rapid systemic elimination. Increasing the metabolism of curcumin or using the adjuvant only is not sufficient to increase the bioavailability of curcumin. The solubility should also be increased in order to improve the bioavailability. Hence, looking at the major issue of bioavailability of curcumin, there is a need to develop a composition of curcumin with combination of ingredients that enhance the absorption of curcumin in blood. Such composition may be further used as a natural cure for treatment of many diseases like arthritis, gastrointestinal disorder, cancer etc.

### SUMMARY OF THE INVENTION

The present invention discloses a curcumin composition to enhance the bioavailability of curcumin in the blood stream. The curcumin composition mainly consists of curcumin mixture and water extract. The curcumin mixture comprises curcumin dry crystals, volatile oil, fixed oil whereas water extract comprises soluble proteins, dietary fibers and carbohydrates extracted from turmeric. The composition also consists of a natural emulsifier isolated from an American tree Quillaja saponaria and lecithin but does not contain any synthetic polymers and is completely derived from the natural components of turmeric. This composition results in increased rate of availability of curcumin in the body fluids as the dietary fibers holds the curcumin for long time, which plays a vital role in prohibiting easy degradation of curcumin inside the tissues. The soluble proteins increases the solubility of curcumin in water thus supporting the increased absorption of curcumin in blood and the essential oil derived from turmeric offers much more dispersibilty to the composition.

The curcumin is extracted from the turmeric using ethanol. The present invention also discloses a method of extraction of curcumin from turmeric.

The present invention also discloses a method of preparation of curcumin composition. The curcumin composition is prepared by mixing the curcumin mixture and water extract in a ratio of 70:30. The mixture is subjected to pre-emulsification and the pre-emulsified mixture is further subjected to micro milling. Finally, the micro milled product is formulated into microcapsules through microencapsulation. The curcumin composition thus produced increases the bioavailability of curcumin by increasing its solubility.

In order to be bioavailable, the nutrient molecule must traverse the inner intestinal wall to gain access to the blood stream. It must pass through the walls of the surface epithelial cells. Cell wall is made of lipids and the molecules, which are fat soluble can easily, pass through them. Curcuminoids are fat soluble and thus they tend to arrive at the cell walls in the form of relatively large, undissolved particles rather than as individual molecules. Such molecular aggregates tend to pass through the epithelial cells but are very poorly absorbed. In case of water soluble molecules, the molecules are readily absorbed as they arrive at the cell wall as individual molecules. However, it is known that the cell walls are lipidic in nature, which means that they tend to repel these water soluble molecules, and thus these molecules find resistance at the cell walls and are thus poorly absorbed. Generally, hydrophilic molecules dissolve easily in the gastrointestinal tract but may be poorly absorbed, where as hydrophobic molecules do not dissolve easily but would be readily absorbed.

The curcumin composition showed bioequivalence with 500 mg curcumin capsules without altering biological or hematological parameters in human. The curcumin composition exhibited anti-aging, antioxidant, anti-inflammatory, wound healing activities and is also effective in boosting the phagocyte mediated immunity suggesting as useful natural therapy for treatment of various diseases.

### BRIEF DESCRIPTION OF DRAWINGS

FIG. 1 illustrates a flow chart of method of extraction of curcumin from turmeric.

FIG. 2 illustrates a flow chart of method for preparation of curcumin composition.

### DETAILED DESCRIPTION OF THE INVENTION

In order to make the matter of the invention clear and concise, the following definitions are provided for specific terms used in the following description.

The term “Curcumin” means the principal curcuminoid found in turmeric, which is a member of the ginger family.

The term “Bioavailability” means one of the principal pharmacokinetic properties of any compound that may be used as drug. It is the measure of presence of drug in the systemic circulation once taken orally in terms of solubility, dispersibilty etc.

The term “Dispersibilty” means to spread widely and uniformly throughout a solid, liquid or gas.

The term “Solubility” means the property of any compound to dissolve in a solid, liquid, or gas to form a homogeneous solution.

The term “Soluble proteins” means proteins with polar charge on them that can dissolve in water as water is also a polar compound.

The term “essential oil of turmeric” means “volatile oil” or “volatile oil of turmeric”, the essential oil of turmeric is a mixture of oils.

The term “antioxidant” means a substance that removes potentially damaging oxidizing agents in a living organism.

The term “anti-aging agent” means a substance that retards the age of a living organism.

The term “anti-inflammatory agent” means a substance which relieves the inflammation i.e. reduces the swelling and pain.

The term “anticancer agent” means a substance that is used for prevention or treatment of cancer.

The present invention discloses a composition of curcumin to overcome the drawback of poor bioavailability exhibited by currently available curcumin compositions. The composition consists of curcumin as the major ingredient along with dietary fiber, soluble protein and essential oil of turmeric, which enhances the bioavailability by increasing solubility of curcumin and augments the utmost utilization of curcumin for its diverse health benefits.

Method of Extraction of Curcumin

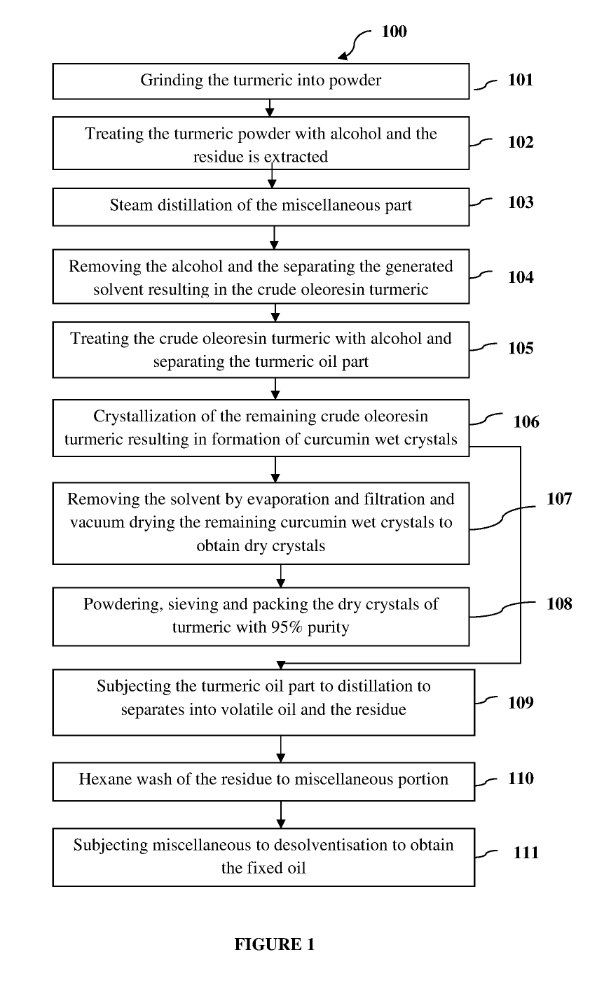

FIG. 1 illustrates the method of extraction of curcumin from turmeric. The curcumin is extracted from turmeric using ethanol. The method 100 includes step 101 of grinding the turmeric into powder. After grinding, the method includes the step 102 of treating the turmeric powder with ethanol, during which, the residue is extracted and step 103 of subjecting the miscellaneous part to steam distillation. After steam distillation, includes the step 104 of removing the ethanol and the separating the generated solvent resulting in the crude oleoresin turmeric. After separating, includes the step 105 of treating the crude oleoresin turmeric with ethanol and separating the turmeric oil part thus obtained. After separating the turmeric oil, includes the step 106 of crystallization of the remaining crude oleoresin turmeric resulting in formation of curcumin wet crystals. After that, includes the step 107 of removing the solvent by evaporation and filtration and vacuum drying the remaining curcumin wet crystals to obtain dry crystals. Finally, the step 108 of powdering, sieving and packing the dry crystals of turmeric which is of 95% purity.

The turmeric oil part separated at step 105 of the above method is subjected to distillation, which separates into volatile oil and the residue 109. The volatile oil is used in preparation of curcumin composition and method includes the step 110 of subjecting the residue to hexane wash. This result in formation of miscellaneous portion. After that, includes the step 111 of subjecting miscellaneous to desolventisation to obtain the fixed oil.

The water extract of turmeric is obtained by the following process. The ground turmeric is treated with water, during which, the residue is extracted and water miscellaneous part is subjected to steam distillation to remove the solvent. The remaining part is the water extract, which consists of turmerin, dietary fiber and carbohydrates.

Composition of Curcumin

The curcumin composition is prepared to increase the bioavailability of curcumin by increasing its solubility. The ingredients of the composition are curcumin mixture and the water extract. The curcumin mixture comprises curcumin dry crystals, volatile oil, fixed oil whereas the water extract comprises soluble protein, dietary fiber and essential oil isolated from turmeric. More specifically, the soluble protein used in the composition is turmerin, which is extracted from turmeric. The dietary fiber rich product obtained from turmeric during hot water extraction is used to maintain the property of rhizome itself in the composition. The soluble protein and dietary fiber holds the curcumin firmly thus preventing its fast elimination from the blood stream. As the soluble proteins are water soluble, the absorption rate of curcumin is increased in water. Another important ingredient is the volatile oil obtained from turmeric via steam distillation. Addition of volatile oil to the composition increases the dispersibilty of curcumin. The composition also consists of a natural emulsifier isolated from an American tree Quillaja saponaria and lecithin but does not contain any synthetic polymers and is completely derived from the natural components of turmeric. The major component curcumin accounts to 60% of the total preparation. While dietary fiber and soluble proteins accounts for 15% each, the rest 10% is contributed by essential oils derived from turmeric. All these components provide curcumin an ‘ambience’ just like in turmeric, which prevents the rapid metabolism of curcumin inside the body resulting in enhanced availability in blood and tissues.

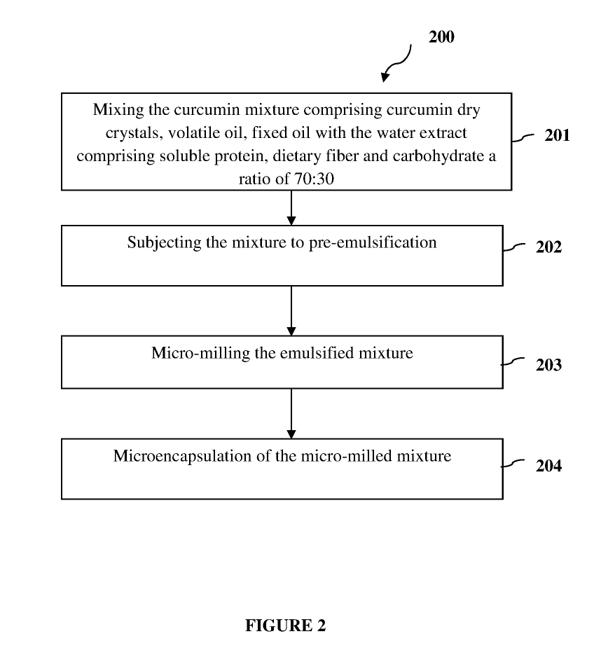

FIG. 2 illustrates the flow chart of method for preparation of curcumin composition. The composition of curcumin is prepared by following method. The method 200 includes step 201 of mixing the curcumin mixture comprising volatile oil, fixed oil with the water extract comprising soluble protein, dietary fiber and carbohydrate in a ratio of 70:30. The natural emulsifier isolated from Quillaja saponaria or lecithin is also added to the composition. After mixing, the method includes the step 202 of subjecting the mixture to pre-emulsification followed by step 203 of micro-milling the emulsified mixture and finally step 204 of microencapsulation. Microencapsulation of the curcumin composition provides protection to the composition, provides sustained release of the curcumin thus increasing the bioavailability of curcumin. The microcapsules of curcumin thus prepared enhance the solubility and the bioavailability of curcumin.

The curcumin composition thus prepared is a combination of polar and non-polar molecules. The non polar molecules includes the essential oil and the polar molecules includes curcumin dry crystals with 95% purity, and finally the water extract of turmeric, mainly containing carbohydrate, dietary fiber and soluble protein turmerin. This polar-non polar composition formulated as microcapsule easily pass through the lipid membrane, as the curcumin is lipidic in nature. Once it is passed through, the polar-non polar matrix makes the curcumin a single molecule, such that the curcumin is easily absorbed and increases the solubility of curcumin thus increasing its bioavailability in the blood stream.

A randomized, open label, balanced, single dose, crossover bioequivalence study was conducted in human volunteers under fasting conditions to check the bio-availability and to compare the single oral bioequivalence of the prepared curcumin composition (test) with 500 mg curcumin capsules (reference). The study was also aimed to monitor the safety and tolerability of a single oral dose of the prepared curcumin composition when administered in healthy human adult volunteer under fasting conditions.

The subjects were dosed either with the prepared curcumin composition (test) or curcumin capsules (reference) in each period as determined by the randomization schedule with a washout period of 14 days between the dosing. The pre-dose sample was collected within 1 hour prior to drug administration. The post-dose blood samples were collected at 1.00, 2.00, 3.00, 4.00, 5.00, 6.00 and 8.00 hours in each study period. A total of 8 (1×5 mL) blood samples were collected for each subject in pre-specified vacuum tubes containing anti-coagulant in each study period. Blood samples were collected from each subject in pre-specified vacuum tubes containing Potassium-Ethylenediaminetetraacetic acid (EDTA) in each study period. The results showed that the prepared curcumin composition is bioequivalent to the 500 mg curcumin capsules and did not result in significant change in biochemical and hematological parameters in human.

The prepared curcumin composition showed a concentration dependent DPPH free radical scavenging activity indicating its antioxidant potential equivalent to ascorbic acid and suggesting that the prepared curcumin composition is a good source of natural antioxidant. The prepared curcumin composition also showed anti-aging activity through hyaluronidase inhibition and anti-inflammatory activity through elastase inhibitory activity in the concentration range between 10 to 100 (μg/ml).

The prepared curcumin composition is also effective in boosting phagocyte mediated immunity by potentiating the phagocyte viability. The prepared curcumin composition exhibited wound healing activity by improving the growth of blood vessels due to presence of angiogenic factors.

We claim:

1. A composition for increasing the bioavailability of curcumin, the composition comprises:
a. a curcumin mixture, wherein the curcumin mixture has curcumin dry crystal, volatile oil, fixed oil and an emulsifier;
b. water extract, wherein water extract has a soluble protein, a dietary fiber and carbohydrate.

2. The composition as claimed in claim 1, wherein a weight of the curcumin mixture and water extract is in a ratio of 70:30.

3. The composition as claimed in claim 1, wherein the emulsifier is selected from a group consisting of a natural emulsifier isolated from an American tree Quillaja saponaria and lecithin.

4. The composition as claimed in claim 1, wherein the soluble protein used is a turmerin extracted from turmeric wherein the turmerin increases the absorption of curcumin.

5. The composition as claimed in claim 1, wherein the volatile oil, fixed oil, the dietary fiber and carbohydrate are isolated from turmeric wherein volatile oil increases the dispersibilty of curcumin.

6. A method for preparing curcumin composition, the method comprising the steps of:
a. grinding turmeric to obtain a powder and treating the obtained turmeric powder with ethanol to convert into a residue and water miscellaneous part;
b. extracting the residue and steam distillation of water miscellaneous part;
c. removing the ethanol after steam distillation and separating water extract from a crude oleoresin turmeric;
d. treating crude oleoresin turmeric with ethanol and separating the turmeric oil thus obtained;
e. crystallization of the residual crude oleoresin turmeric resulting in formation of curcumin wet crystals;
f filtering and vacuum drying of the curcumin wet crystals to convert into curcumin dry crystals;
g. powdering, sieving and packing the curcumin dry crystals;
h. distillation of the turmeric oil separated in Step of crystallization to convert into volatile oil and the residue;
i. washing the residue using hexane to form a miscellaneous portion and desolventisation of the miscellaneous portion to obtain fixed oil;
j. mixing the curcumin mixture comprising the curcumin dry crystals, volatile oil, fixed oil and water extract comprising a soluble protein, a dietary fiber and carbohydrate;
k. pre-emulsification of the curcumin mixture and water extract; and
l. micro-milling the emulsified curcumin mixture and microencapsulation of the micro-milled curcumin mixture into one or more microcapsules.

7. The method as claimed in claim 6, wherein mixing the curcumin mixture and water extract is in a ratio of 70:30.

8. The method as claimed in claim 6, wherein the dry crystals is of 95% purity.
